(19) United States
(12) Patent Application Publication
(10) Pub. No.: US 2010/0028268 A1
(43) Pub. Date: Feb. 4, 2010

(54) Compositions and Methods For Treating Infectious Bronchitis

(75) Inventors: Richard Rosenbloom, North Wales, PA (US); Timothy Cummings, Mississippi State, MS (US); Michael Petteruti, Hilltown, PA (US)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(73) Assignee: THE QUIGLEY CORPORATION, Doylestown, PA (US)

(21) Appl. No.: 12/513,979

(22) PCT Filed: Dec. 14, 2007
(86) PCT No.: PCT/US07/87551

§ 371 Date: May 7, 2009

Related U.S. Application Data
(60) Provisional application No. 60/869,991, filed on Dec. 14, 2006.

(51) Int. Cl. A61K 9/12; A61K 36/82

(52) U.S. Cl. 424/45; 424/729

(57) ABSTRACT

A prophylactic treatment for infectious bronchitis as well as a treatment of infectious bronchitis. The methods comprise the step of administering to a bird, an amount of a composition having a first ingredient obtainable from turmeric; a second ingredient obtainable from green tea; an third ingredient obtainable from ginger; and an acceptable carrier. Compositions in accordance with the invention may be employed for the purpose of reducing the incidence of contracting infectious bronchitis or reducing the transmissivity of infectious bronchitis.

### BACKGROUND OF THE INVENTION

1. Field of the Invention

The present invention relates methods for treating or reducing the incidence of infectious bronchitis. More particularly, the present invention relates to methods for treating, or reducing the incidence of infectious bronchitis in birds as well as reducing the transmissivity of infectious bronchitis.

2. Description of the Related Technology

Infectious bronchitis is an acute and highly contagious respiratory disease affecting chickens, caused by a coronavirus. Considered one of the most contagious poultry diseases, the virus is capable of spreading by direct contact with infected chickens or contaminated surfaces such as poultry crates, equipment and general premises. Infectious bronchitis has a high mortality rate, especially in young poultry. Infected chickens experience severe respiratory symptoms such as gasping, coughing, lower egg production due to lesions to the oviduct, and/or induced stress as a result of the infection. Moreover, infectious bronchitis may stimulate latent viral or bacterial infections and may give rise to severe economic losses, especially in the broiler field. To date, there is no known, effective treatment for this disease.

To combat infectious bronchitis, it is well known to utilize vaccines derived from inactivated viruses and/or live viruses. A number of problems, however, frequently occur as a result of administering inactivated and/or live viruses. In some cases, a loss of immunogenic properties may occur after inactivation of the infectious bronchitis virus with inactivation agents such as formaline and ultra violet light (M. S. Hofstad, Diseases of Poultry, Biester and Schwarte, Iowa University, Press. Ames. (1965), 615). Healthy chickens may also be killed or become diseased by primary vaccination with live, non-attenuated or slightly attenuated virus vaccines. A special danger exists for animals of less than 2 or 3 weeks old and chickens which are currently, or will shortly, begin laying eggs.

Another form of vaccination utilizes modified live virus vaccines, viruses having undergone 25 or more embryo passages to reduce their pathogenicity, such as those derived from the Massachusetts type and more particularly the IBV W 48, M 41 and 82828 strains, besides the Connecticut isolates, e.g. the A 5968 strain. The immunizing capacity of these viruses, however, is specific to a particular viral infectious bronchitis strain. Therefore the vaccine is unable to immunize birds against antigenic variations of the virus (Archiv fur die Gesamte Virusforschung 34, p. 32 (1971) and Cunningham C. H. Develop. Biol. Standard, 33 p. 311 (1976)) and are unable to effectively prevent outbreaks (Avian Diseases, Vol. 20, No. 1, pages 42 and 177 (1976) and Avian Diseases, Vol. 19, No. 2, pages 323 and 583 (1975)). Moreover, vaccination of poultry using combined vaccines derived from IBV strains of different serotypes corresponding with the IBV types decrease the immunogenic properties of the respective starting viruses caused by mutual interaction (Am. J. Vet. Res. 36, 4, 524 and 525 (1965) and Avian Diseases 12, 577 (1968)).

As a result of these common problems, investigations have been conducted to develop non-virus based methods of treatment. Although there exist a few references, such as U.S. publications nos. 2003/0185912 and 2005/0147697, that address the antiviral properties of a composition composed of turmeric, green tea, ginger and horseradish, such publications do not disclose a method for treating infectious bronchitis.

Therefore, a need exists for a method for treating, reducing the incidence of, or reducing the transmissmivity of infectious bronchitis.

### BRIEF DESCRIPTION OF THE DRAWINGS

FIG. 1 shows a graph of dilution log versus cell concentration depicting the affect of Composition 1 on the viability of infectious bronchitis virus (IBV) strain Beaudette in VERO E6 cells.

FIG. 2 shows a graph of dilution log versus cell concentration depicting the affect of Composition 1 on the viability of IBV strain Beaudette in embryonating eggs.

FIG. 3 shows on the left panel, cells showing no cytopathic effects (CPE) following exposure to IBV treated with a 1×10⁻³ dilution of Composition 1, and on the right panel, cells with CPE following exposure to IBV treated with a 1×10⁻³ dilution of the placebo.

### SUMMARY OF THE INVENTION

The invention is directed to methods for the treatment of infectious bronchitis, for reducing the incidence of contracting infectious bronchitis and for reducing the transmissivity of infectious bronchitis.

In a first aspect, the invention relates to a method for the prophylactic use of a composition to reduce the incidence of contracting and/or transmitting infectious bronchitis. The method comprises the steps of administering to a bird that has been, might be, or will be, exposed to infectious bronchitis virus, an effective amount of a composition including a first ingredient obtainable from turmeric and a second ingredient obtainable from green tea. The amount of the composition is effective, when administered, to reduce the incidence of contracting and/or transmitting the infectious bronchitis virus.

In a second aspect of the invention, the composition including a first ingredient obtainable from turmeric and a second ingredient obtainable from green tea may be used to treat a bird with infectious bronchitis by administering an effective amount of the composition to treat infectious bronchitis.

A third aspect of the invention relates to an aerosol, splay, mist or liquid composition including a first ingredient obtainable from turmeric and a second ingredient obtainable from green tea; and an acceptable vehicle. The aerosol, spray, mist or liquid composition may be administered as a treatment for infectious bronchitis or administered prophylactically to birds using any conventional techniques for which an aerosol or liquid composition is suitable, e.g. spraying or misting of birds.

In a fourth aspect, the present invention relates to animal feeds which include a first ingredient obtainable from turmeric and a second ingredient obtainable from green tea.

These and various other advantages and features of novelty that characterize the invention are pointed out with particularity in the claims annexed hereto and forming a part hereof. However, for a better understanding of the invention, its advantages, and the objects obtained by its use, reference should be made to the accompanying descriptive matter, in which there is described a preferred embodiment of the invention.

### DETAILED DESCRIPTION OF THE PREFERRED EMBODIMENTS

In one aspect, the present invention relates to a method for the prophylactic use of a composition to reduce the instance of contracting and transmitting the infectious bronchitis virus (IBV). The method comprises the steps of administering to a bird that has been, or will be, exposed to infectious bronchitis, an amount of a composition having a first ingredient obtainable from turmeric; a second ingredient obtainable from green tea; and, optionally, an acceptable carrier. The amount of the composition is effective, when administered, to reduce the incidence of contracting and/or transmitting infectious bronchitis.

In another aspect, the present invention relates to a method for treatment of a bird infected with infectious bronchitis by administering an effective amount of a composition having a first ingredient obtainable from turmeric; a second ingredient obtainable from green tea; and, optionally, an acceptable carrier. The amount of the composition is effective, when administered, to treat infectious bronchitis virus.

The composition for use in the methods of the present invention may include a first ingredient obtainable from turmeric and a second ingredient obtainable from green tea. Ingredients obtainable from ginger and/or horseradish may also be included as optional additional active ingredients of the composition of the present invention.

As used herein, the term “acceptable” means a component that is suitable for use with birds without undue adverse side effects (such as toxicity, irritation, and allergic responses), commensurate with a reasonable risk/benefit ratio.

Further, as used herein, the term “safe and effective amount” refers to the quantity of a component, which is sufficient to yield a desired therapeutic response without undue adverse side effects (such as toxicity, irritation, or allergic responses), commensurate with a reasonable risk/benefit ratio when used in the manner described herein.

The term “inhibiting”, as used herein, refers to reducing or preventing further growth of an infectious bronchitis viral strain, or preventing the infectious bronchitis viral strain from attaching to normal cells, and/or the elimination of some or all of the infectious particles from the human or animal being treated. Suitable methods for determining viral inhibition are discussed in the examples.

The term “transmissivity” or “transmitting” as used herein refers to the transfer of a microbe from one host to another.

All active compounds used in the present invention may be obtained from other sources, if available. Thus, the phrase “which can be obtained from” or the phrase “which may be obtained from” is meant to encompass compounds or compositions that are obtainable from turmeric, green tea, ginger or horseradish, and therefore encompasses synthetic forms of the same compounds and/or compositions as well as the same compounds and/or compositions obtained from other sources.

In a first embodiment, the composition of the present invention includes a first ingredient obtainable from turmeric, and a second ingredient obtainable from green tea, in a safe and effective amount to provide one or more of the beneficial effects described herein.

The first ingredient of the composition of the present invention may be obtained from turmeric, and is used in a safe and effective amount to provide one or more of the beneficial effects described herein. Turmeric (Curcuma longa), or Haldi in Hindi, is used very widely as medicine as well as a common ingredient in Indian cooking. The rhizome of turmeric is used in medicine and food as a fine powder.

The yellow pigment of the rhizome of turmeric is composed of three compounds known as curcuminoids. The three curcuminoids are curcumin (diferuloylmethane), desmethoxycurcumin (hydroxycinnamoyl feruloylmethane), and bis-desmethoxycurcumin (dihydroxydicinnamoyl methane) (see Drug Analysis, Chromatography and Microscopy, p. 169, Ann Arbor Science Inc., 1973). The essential oils of turmeric (Curcuma longa) are primarily composed of the following compounds: d-camphor (about 1%), cyclo-isoprenemyrcene (about 85%), and p-tolylmethylcarbinol (about 5%), (see E. Gunther, The Essential Oil, pp. 123-4, Van Nostrand Co., 1955).

The ingredient of the composition of the present invention, obtained from turmeric, preferably includes curcuminoids, such as curcumin (diferuloylmethane), desmethoxycurcumin (hydroxycinnamoyl feruloylmethane), and bis-desmethoxycurcumin (dihydroxydicinnamoyl methane), and mixtures of two or more of these curcuminoids.

Methods for isolating curcuminoids from turmeric are known (see Janaki and Bose, An Improved Method for the Isolation of Curcumin From Turmeric, J. Indian Chem. Soc. 44: 985, 1967). Alternatively, curcuminoids for use in the present invention can be prepared by synthetic methods.

The ingredient, which can be obtained from of turmeric, can be incorporated into the composition of the present invention in a variety of different forms. Those different forms preferably include extracts of turmeric such as turmeric extracts including turmeric powder extracts, turmeric fluid extracts, Aquaresin® turmeric, Oleoresin® turmeric, one or more the curcuminoid compounds, and turmeric powder, parts of, or whole plants of turmeric, tinctures thereof, and mixtures thereof. More preferably, the first ingredient obtainable from turmeric is a turmeric extract.

When the ingredient obtainable from turmeric is used, each gram of the composition of the present invention preferably contains about 0.001 mg to about 20 mg of an ingredient obtainable from turmeric such as turmeric powder extract. Most preferably, each gram of the compositions contains about 0.01 mg to about 15 mg of an ingredient obtainable from turmeric such as turmeric powder extract. These ranges are based on the use of Turmeric Extract 95%, ex. Pharmline, Inc. in the ingested formulation and Turmeric Root Extract (Oleoresin® Turmeric), ex. Kalsec, Inc., Kalamazoo, Mich., in the spray formulation.

The second ingredient of the composition of the present invention may be obtained from green tea. The second ingredient obtained from green tea may have an antioxidant effect. Green tea is the dried leaves and leaf buds of the shrub Camellia sinensis. It is mainly produced in China and Japan. Dried tea leaves are composed mainly of phytochemicals known as polyphenols (about 36%), principally flavonols (including catechins), flavonoids, and flavondiols. The leaves also contain plant alkaloids (about 4%), including caffeine, theobromine and theophylline.

The pharmacological activities of green tea are mainly due to its active compounds. The active compounds of green tea useful in the present invention include, but are not limited to, flavonols, catechins, flavonoids, flavondiols, plant alkaloids, caffeine, theobromine, theophylline, phenolic acids, proteins, carbohydrates, and minerals.

The second ingredient which may be obtained from green tea, can be included in the composition in the form of green tea powder, green tea extracts such as green tea powder extracts, green tea fluid extracts, and one or more active compounds of green tea, part of, or whole green tea plants, green tea leaves, tinctures thereof, or mixtures thereof. Preferably, the second ingredient of the composition of the present invention is selected from green tea leaves, green tea powder and green tea extract. More preferably, the second ingredient of the composition of the present invention is green tea extract.

Each gram of the composition of the present invention preferably contains about 0.001 mg to about 20 mg of an ingredient obtainable from green tea such as green tea extract. Most preferably, each gram of the composition contains about 0.01 mg to about 15 mg of an ingredient obtainable from green tea such as green tea extract. These ranges use, as a baseline, the use of Green Tea, ex. Stryker Botanics in the ingested formulation and Green Tea Extract, ex. Phytoway, Inc., Chang Sha, P.R. China, in the spray formulation.

An optional ingredient of the composition of the present invention may be obtained from ginger, in a safe and effective amount. Native to southern Asia, ginger is a 2- to 4-foot perennial that produces grass-like leaves up to a foot long and almost an inch wide. Ginger root, as it is called in the grocery store, actually consists of the underground stem of the plant, with its bark-like outer covering scraped off.

The active compounds of ginger which may be employed in the present invention include, but are not limited to, 1,8-cineole, 10-dehydrogingerdione, 10-gingerol, 6-gingerdione, 6-gingerol, 6-shogaol, 8-.beta.-17-epoxy-.lambda.-trans-12-ene-15,16-diol, 8-gingerol, 8-shogaol, 9-oxo-nerolidol, acetaldehyde, acetic acid, alanine, .alpha.-linolenic-acid, .alpha.-linolenic acid, .alpha.-phellandrene, .alpha.-piene, .alpha.-terpinene, .alpha.-terpineol, .alpha.-zingiberene, ar-curcumene, arginine, ascorbic acid, asparagine, .beta.-bisabolol, .beta.-carotene, .beta.-elemene, .beta.-eudesmol, .beta.-ionone, .beta.-myrcene, .beta.-phellandrene, .beta.-pinene, .beta.-selinene, .beta.-sesquiphellandrene, .beta.-sitosterol, .beta.-thujone, bornyl-acetate, boron, caffeic acid, calcium, camphene, camphor, capric acid, caprylic acid, capsaicin, caryophyllene, chavicol, chlorogenic acid, chromium, citral, citronellal, citronellal, cobalt, copper, cumene, curcumin, cystine, delphinidin, .delta.-cadinene, elemol, ethyl acetate, ethyl-myristate, farnesal, farnesene, ferulic acid, furfural, .gamma.-aminobutyric acid, .gamma.-terpinene, geranial, geraniol, geranyl-acetate, gingerenone, glutamic acid, glycine, hexahydrocurcumin, histidine, isogingerenone-B, isoleucine, kaempferol, lecithin, limonene, linoleic acid, magnesium, manganese, methionine, mufa, myrecene, myricetin, myristic acid, neral, nerol, nerolidol, niacin, nickel, oleic acid, oxalic acid, p-coumaric acid, p-cymene, p-hydroxy-benzoic acid, palmitic acid, pantothenic acid, paradol, patchoulic alcohol, phenylalanine, quercetin, riboflavin, selenium, shikimic-acid, terpinen-4-ol, thiamin, tryptophan, vanillic acid, vanillin, zinc, and zingerone. Also, mixtures of two or more of these active compounds may be employed.

Ginger, can be incorporated in the composition of the present invention in many different forms including extracts such as ginger extracts including ginger powder extracts, ginger fluid extracts, ginger powder including ginger root powder, Aquaresin® ginger, oleoresin ginger, and one or more active compounds of ginger, parts of, or whole ginger plants, tinctures thereof, and mixtures thereof. Preferably, the optional ingredient of the composition of the present invention is selected from ginger extract, and ginger powder.

Each gram of the composition of the present invention preferably contains about 0.001 mg to about 30 mg of an ingredient obtainable from ginger such as ginger extract. Most preferably, each gram of the composition contains about 0.01 mg to about 20 mg of an ingredient obtainable from ginger such as ginger extract. These ranges use, as a baseline, the use of Ginger Root Powder, ex. Stryka Botanics in the ingested formulation and Ginger Extract K (Aquaresin® ginger), ex. Kalsec, Inc. of Kalamazoo, Mich. in the spray formulation.

The amounts of various ingredients are given herein in terms of one form of the ingredient, i.e. ginger root extract. If that ingredient is present in another form, then the amount to be employed is that amount which will provide the same amount of the one or more active compounds as the amount of that ingredient given herein.

Also, the composition of the present invention may include one or more ingredients obtainable from horseradish, in a safe and effective amount to provide one or more of the beneficial effects described herein. The optional ingredient obtainable from horseradish may include extracts from the Cochlearia Armoracia. The ingredient obtainable from horseradish may be in the form of a horseradish extract, such as, for example horseradish powder extracts, horseradish liquid extracts and horseradish root extracts such as horseradish oil. Horseradish contains volatile oils that are similar to those found in mustard. These include glucosinolates (mustard oil glycosides), gluconasturtiin, and sinigrin, which yield allyl isothiocynate when broken down in the stomach. The compositions of the present invention preferably contain from about 0.0001 mg to 10 mg of an ingredient obtainable from horseradish such as horseradish oil, per gram of the composition, and, more preferably, the compositions of the present invention contain from 0.001 mg to 5 mg of an ingredient obtainable from horseradish such as horseradish oil, per gram of the composition.

Ethanol, propylene glycol and glycerin and various combinations thereof, may be optionally included in liquid compositions of the present invention, up to about 10 percent by weight of the total as optional ingredients. Most preferably, up to about 10 percent per total weight ethanol is added as an optional ingredient. Even more preferable, 2.5 to 7 percent ethanol is added.

Preferably, the ingredients described above, that may be derived from turmeric and green tea and, optionally, ginger and/or horseradish, make up from about 0.001 to about 90% by weight of the total composition. More preferably, the main ingredients will make up about 0.01 to about 20% by weight of the total composition. Most preferably, the main ingredients make up about 1 to about 10% by weight of the total composition.

The non-carrier ingredients of the composition, including the ingredients obtainable from turmeric, ginger, and green tea as discussed above, can be increased or decreased proportionally in the composition of the present invention depending on the amount of carrier used in the composition, without substantially affecting the effectiveness of the composition for its intended use.

The plant extracts, e.g., turmeric extract, ginger extract, green tea extract and horseradish extract that may be used in the compositions of the invention, may be produced using common extraction procedures. Alternatively, the extracts may be purchased from commercial sources such as the Kalsec, Inc. of Kalamazoo, Mich.

The processes for the preparation of pharmacologically or biologically active plant extracts in a convenient, administrable dosage form from any of the plants mentioned above are well known in the art.

The composition of the present invention may be used to prevent the infectivity and transmissivity of various strains of the infectious bronchitis virus among birds to thereby reduce the incidence of infection among birds. The composition may also be used as a therapeutic composition to treat infectious bronchitis and thereby alleviate symptoms associated with infectious bronchitis.

A safe and effective amount of the composition of the present invention may be administered to a bird that has been or will be exposed to infectious bronchitis, to reduce the incidence of contracting said illness, relative to a bird that has been or will be exposed to the infectious bronchitis virus.

Preferably, the composition of the present invention may be formulated in any acceptable dosage form including, but not limited to animal feeds such as bird feed, powders, sprays, nasal sprays, nasal drops, suspensions, solutions, injections or any standard form for mass inoculation. The composition of the present invention may also be administered in the form of a nutritional supplement, in which case the composition of the invention may be the nutritional supplement or may form a part of a nutritional supplement containing additional ingredients.

Tablets in this invention may differ in shape, size and manufacturing technique. In the case of tablets, for oral use, the acceptable carrier may further include lactose and corn starch. Lubricating agents may also be added to the tablets, including, for example, magnesium stearate, sodium lauryl sulfate and talc. Tablets may also contain excipients such as sodium citrate, calcium carbonate and calcium phosphate. Disintegrants such as starch, alginic acid and complex silicates may also be employed. Tablets may also include binding agents such as polyvinylpyrrolidone, gelatin, PEG-8000 and gum acacia.

Alternatively, the composition of the present invention may be formulated in liquid form, such as syrups, solutions, liquid formulations, mists or sprays, with a solvent or dispersant such as water, or other liquids and optionally in a pharmaceutically acceptable carrier, for repeated delivery of the composition to oral and oropharyngeal mucous membranes over a sustained period of time. Preferably, the treatment time is about 5 to 60 minutes, and more preferably about 20 to 30 minutes, so as to permit a prolonged contact of the composition with mouth, nasopharnyx and throat tissues. Alternatively, such formulations can be in a concentrated form suitable for dilution with water or other materials prior to use.

The composition of the present invention may also be formulated with an acceptable carrier. The acceptable carrier may include, but is not limited to: (a) glycerin; (b) ethanol; (c) phospholipids; (d) MCT oil; (e) water; and (f) suitable relatively insoluble excipients including starches, cellulose, cyclodextrins, silica and lipids/fats.

The composition may also be formulated in chewable forms, such as a component of animal feeds and/or as a food additive. The composition of the invention may alternatively be formulated in capsule form, with or without diluents. For capsules, useful diluents include lactose and dried corn starch. When suspensions are employed, emulsifying and/or suspending agents may be employed in the suspensions. In addition, solid compositions including one or more of the ingredients of the lozenges described above may be employed in soft and hard gelatin capsules.

The composition of the present invention may also be formulated into an aerosol or inhalant composition. Such a composition may be prepared using well-known techniques. For these types of formulations, suitable carriers may include the following ingredients: saline with one or more preservatives, absorption promoters to enhance bioavailability, fluorocarbons, and/or conventional solubilizing or dispersion agents.

The composition may also be administered using any known standard delivery means. The composition may be formulated as a water or solution additive. Moreover, the composition may be administered in ovo.

Other materials, which may optionally be included in the composition of the present invention, include resveratrol (trihydroxystilbene), inositol, other B-complex vitamins, and additional anti-inflammatories. Also, ingredients such as sweeteners, flavorants, coloring agents, dyes, preservatives, emulsifying agents, suspending agents, melting agents, excipients, demulcents and solvents or diluents such as water, ethanol, propylene glycol, glycerin and various combinations thereof, may be included in the composition of the present invention.

Reducing or preventing transmission relates to preventing or reducing the spread of a microbe from one bird (infected) to another bird (non-infected). Some birds may be considered carriers of the infection. Carriers are individuals who actively shed infectious bronchitis microbes but do not suffer from an acute infection. These carriers may be said to be persistently (or chronically) infected with a strain of infectious bronchitis. In addition to the persistently infected shedder, other infective birds may be those which are actively infected, and particularly those in the early or late stages of an acute infection. One aspect of the invention relates to administering to a bird infected with a strain of infectious bronchitis, the composition of the present invention, to prevent the spread of the disease to other birds.

Prophylactic treatment is aimed at a bird that will soon be exposed to the infectious bronchitis virus or has recently been exposed to the infectious bronchitis virus for the purpose of reducing the instance of active infection. Such prophylactic treatment may be effective either alone or in addition to a vaccine. The prophylactic treatment of the present invention may also be used against viral strains of infectious bronchitis for which there is not yet a vaccine available.

The invention also relates to a method of treating a bird infected with infectious bronchitis to treat the disease by, for example, reducing the duration, fatality rate, or adverse effects of the disease. The present invention may reduce, treat, alleviate or at least partially prevent at least one symptom or adverse effect of viral infection. Such symptoms include lack of energy, decreased egg production, soft shelled eggs, sinus swelling, nasal discharge, coughing, sneezing, chirping, diarrhea, tracheal rattling, wet eyes and fluid buildup in the abdomen.

The composition may be administered to any member of the avian species, which includes the common commercial poultry birds: chicken, turkeys, ducks and geese, less commonly, the ostrich as well as other bird species that are commonly kept as house pets, for example canaries and parrots.

The composition may be administered by directly spraying the composition into the nasal passage of the bird, spraying the composition into the oral cavity of the bird or the composition may be administered by creating a mist to which the birds are exposed. Thus, the composition may be given prophylactically to act in a virucidal or virustatic manner. Alternatively, the composition may be used to reduce the transmissivity of the virus.

The effective amount of the composition will vary depending on such factors as the patient being treated, the particular mode of administration, the activity of the particular active ingredients employed, the age, bodyweight, general health, sex and diet of the bird, time of administration, rate of excretion, the particular combination of ingredients employed, the total content of the main ingredient of the composition, and the severity of the illness or symptom. It is within the purview of one of ordinary skill in the art to account for these factors.

The composition may be administered about 1 to about 15 times per day, as needed, more preferably, about 2 to about 12 times per day, as needed, or most preferably, about 6 to about 10 times per day, as needed. The composition of the present invention may be administered in any acceptable dosage form, as described above, including, but not limited to, tablets, capsules, powders, oral sprays, nasal sprays, chewable compositions, suspensions, solutions and through in ovo administration.

Each dosage of the composition contains a safe and effective amount of the composition of the present invention. An effective amount for each therapeutic administration contains a total of about 0.001 milligram to about 1 gram of the ingredients, which may be obtained from turmeric and green tea. More preferably, an effective amount of the composition for each therapeutic administration contains a total of about 0.01 milligram to about 0.5 gram of the ingredients which may be obtained from turmeric and green tea.

When the composition is administered as a feed or water additive, the amount of the active ingredients in the feed or water additive may range from about 0.01 to 50 weight percent of the total feed composition. In a preferred embodiment, the active ingredients constitute about 0.1 to about 30 weight percent of the total feed composition, and in a most preferred embodiment, the active ingredients constitute about 1 to about 20 weight percent of the total feed composition. The active ingredients may comprise the ingredient from turmeric, the ingredient from green tea, the ingredient from ginger and/or the ingredient from horseradish.

When the composition is administered as a spray, the amounts each of the active ingredients may be reduced as the spray composition delivers the active ingredients more directly to the location where they are needed, as compared to a lozenge or capsule for example. The composition may be diluted to any desired concentration with the addition of water or another suitable diluent; the diluted composition may contain anywhere from about 0.1% to about 99.999% by weight water or other diluent, more preferably about 10% to about 40% water or other diluent by weight, and most preferably 10% to about 30% water or other diluent by weight.

The following preferred ranges define compositions according to the invention that are suited for administration in a spray formulation according to the methods of the invention.

Each gram of the composition administered in a spray according to the methods of the present invention preferably contains about 0.001 mg to about 12 mg of a turmeric extract such as soluble oleoresin turmeric. Most preferably, each gram of the composition contains about 0.01 mg to about 9 mg of a turmeric extract such as soluble oleoresin turmeric. Each gram of the composition administered in a spray according to the methods of the present invention preferably contains about 0.001 mg to about 20 mg of a green tea extract such as green tea leaf extract. Most preferably, each gram of the composition contains about 0.01 mg to about 15 mg of a green tea extract such as green tea leaf extract.

Each gram of an optional embodiment of a composition administered in a spray according to the methods of present invention preferably contains about 0.001 mg to about 10 mg of a ginger extract such as Aquaresin® ginger. Most preferably, each gram of the composition contains about 0.01 mg to about 7 mg of a ginger extract such as Aquaresin® ginger.

Optionally, each gram of the composition also contains from about 0.0001 mg to about 1 mg of horseradish root extract, more preferably about 0.001 mg to about 2 mg of horseradish root extract and most preferably about 0.5 mg to about 1 mg of horseradish root extract.

An effective amount of the composition may also be used to disinfect and/or sterilize any equipment used to administer the composition to the birds so as to inactivate some or all of any strain of the IBV located on the equipment. The composition may be topically applied to any equipment or machine surface to disinfect the instrument.

The invention will be further illustrated by the examples given below which are not to be construed as limiting the invention in any way. The scope of the invention is to be determined by the claims appended hereto.

### EXAMPLES

### Example 1

A Suitable Formulation - Composition 1

| | | | | Actual |
|---|---|---|---|---|
| Component | Target (wt %) | Target (g) | Actual (wt g) | (wt %) |
| Turmeric | 0.6466 | 0.6466 | 0.6952 | 0.6943 |
| Oleoresin ® | | | | |
| Ginger | 0.6840 | 0.6840 | 0.6826 | 0.6817 |
| Oleoresin ® | | | | |
| Horseradish Oil | 0.063120 | 0.0631 | 0.0631 | 0.0630 |
| Green tea, | 0.4619 | 0.4619 | 0.4646 | 0.4640 |
| powered | | | | |
| extract | | | | |
| Glycerin | 46.5723 | 46.5723 | 46.6322 | 46.5701 |
| Ethanol | 5.0000 | 5.0000 | 5.0157 | 5.0090 |
| Phospholipids | 0.5000 | 0.5000 | 0.5093 | 0.5086 |
| MCT oil | 5.0000 | 5.0000 | 5.0033 | 4.9966 |
| Water | 41.0721 | 41.0721 | 41.0673 | 41.0126 |
| Total | 99.9981 | 100.0000 | 100.1333 | 100.0000 |

The formulation may be diluted by a factor of 1-1300 with water or another suitable diluent to provide more dilute compositions for use in a variety of applications such as spraying, misting, as an aerosol or as a liquid formulation.

### Example 2

A study of the safety and tolerability of Composition 1, disclosed in Example 1, to treat infectious bronchitis in chickens using various dosages and routes of administration was performed. The results demonstrated that the Composition 1 is an effective and suitable liquid additive to poultry water supply, liquid additive to a nasal drop formulation or solid additive to poultry feed.

132 White Leghorn chickens, approximately 7 days old, were separated into 11 groups, administered various forms and concentrations of Composition 1 corresponding to Tables 1(a)-1(c) and subsequently exposed to the infectious bronchitis virus.

TABLE 1(a)

| | No. of | Chicken | Route of | Dose/ |
|---|---|---|---|---|
| Group | Chickens | Numbers | Administration | Concentration |
| 1 | 12 | 1-12 | feed (continuous) | high |
| 2 | 12 | 13-24 | feed (continuous) | med |
| 3 | 12 | 25-36 | feed (continuous) | low |
| 4 | 12 | 37-48 | water (continuous) | high |
| 5 | 12 | 49-60 | water (continuous) | med |
| 6 | 12 | 61-72 | water (continuous) | low |
| 7 | 12 | 73-84 | feed & water | high |
| | | | (continuous) | |
| 8 | 12 | 85-96 | feed & water | med |
| | | | (continuous) | |
| 9 | 12 | 97-108 | feed & water | low |
| | | | (continuous) | |
| 10 | 12 | 109-120 | control (none) | — |
| 11 | 12 | 121-132 | nasal drop | — |
| Spare bird | 12 | n/a | n/a | — |
| supply | | | | |
| Total | 144 | | | |
| Birds | | | | |

TABLE 1(b)

| | Route of | Dose/ | Dosing | Dosing |
|---|---|---|---|---|
| Group | Administration | Concentration | Days | Duration |
| 1 | feed (continuous) | high | 1-4 | ad libitum |
| 2 | feed (continuous) | med | 1-4 | ad libitum |
| 3 | feed (continuous) | low | 1-4 | ad libitum |
| 4 | water (continuous) | high | 1-4 | ad libitum |
| 5 | water (continuous) | med | 1-4 | ad libitum |
| 6 | water (continuous) | low | 1-4 | ad libitum |
| 7 | feed & water | high | 1-4 | ad libitum |
| | (continuous) | | | |
| 8 | feed & water | med | 1-4 | ad libitum |
| | (continuous) | | | |
| 9 | feed & water | low | 1-4 | ad libitum |
| | (continuous) | | | |
| 10 | control (none) | — | 1-4 | ad libitum |
| 11 | nasal drop | — | 1-4 | 1 drop per |
| | | | | nostril 4 ×/day |
| Spares | n/a | — | none | none |

TABLE 1(c)

| | | | | | Total Feed | |
|---|---|---|---|---|---|---|
| | | Route of | Dose/ | Days | Consumption per | % drug |
| Group | No. of Birds | Administration | Concentration | Dosing | study period | mixture |
| 1 | 12 | feed | high | 4 | 960 | 0.2 |
| 2 | 12 | feed | med | 4 | 960 | 0.12 |
| 3 | 12 | feed | low | 4 | 960 | 0.02 |
| 4 | 12 | water | high | 4 | 960 | 0 |
| 5 | 12 | water | med | 4 | 960 | 0 |
| 6 | 12 | water | low | 4 | 960 | 0 |
| 7 | 12 | feed & water | high | 4 | 960 | 0.2 |
| 8 | 12 | feed & water | med | 4 | 960 | 0.12 |
| 9 | 12 | feed & water | low | 4 | 960 | 0.02 |
| 10 | 12 | control (none) | — | 4 | 960 | 0 |
| 11 | 12 | nasal drop | — | 4 | 960 | 0 |
| Total | 132 | | | SUB-TOTALS | 10560 | |
| Birds | | | | | | |
| | | | | | 20 gms/ | |
| | | | | | day/bird | |
| | | | | | Total Feed | |
| | | | | | 10560 | |

| | | | Water | | | |
|---|---|---|---|---|---|---|
| | | | Consumption | | | |
| | Amount Drug | Amount pure | entire study | % drug | Amount Drug | Amount pure |
| Group | Required (gms) | feed required | period (ml) | mixture | Required (ml) | water required |
| 1 | 192 | 768 | 1920 | 0 | 0 | 1920 |
| 2 | 115.2 | 844.8 | 1920 | 0 | 0 | 1920 |
| 3 | 19.2 | 940.8 | 1920 | 0 | 0 | 1920 |
| 4 | 0 | 960 | 1920 | 0.2 | 384 | 1536 |
| 5 | 0 | 960 | 1920 | 0.12 | 230.4 | 1689.6 |
| 6 | 0 | 960 | 1920 | 0.02 | 38.4 | 1881.6 |
| 7 | 192 | 768 | 1920 | 0.2 | 384 | 1536 |
| 8 | 115.2 | 844.8 | 1920 | 0.12 | 230.4 | 1689.6 |
| 9 | 19.2 | 940.8 | 1920 | 0.02 | 38.4 | 1881.6 |
| 10 | 0 | 960 | 1920 | 0 | 0 | 1920 |
| 11 | 0 | 960 | — | — | 14 | — |
| Total | 652.8 | 9907.2 | 19200 | | 1319.6 | 17894.4 |
| Birds | | | | | | |
| | | | 40 ml/ | | | |
| | | | day/bird | | | |
| | Drug subtotal | pure feed | | | Drug subtotal | pure water |
| | (gms) | subtotal | Total Water | | (ml) | subtotal |
| | 652.8 | 9907.2 | 19200 | | 1319.6 | 17894.4 |

The chickens were housed three in each cage. The cages were maintained at about 85 degrees Fahrenheit and with a relative humidity at 65%. They were provided with 16 hours of continuous, incandescent lighting, followed by 8 hours of darkness each day.

The chickens were monitored to determine their tolerability to and the toxicity of Composition 1. Quantities of water and feed consumed by each group were assessed and individual chicken weights were routinely weighed and measured.

For chickens which were administered Composition 1 in the form of nasal drops, the nasal drops were administered, one (1) drop per each nostril, four (4) times daily, for 4 days of dosing, in alignment with the feed and water dosing groups. Drops may be administered twice in the morning with each administration being approximately 1 hour apart, and twice in the afternoon/evening also with each administration being approximately 1 hour apart. This nasal dosing schedule is flexible so that a total of 4 drops per each nostril can be administered per day, yet consecutive morning or afternoon doses are not spaced closer than 1 hour apart. Nasal drops were administered using a standard bottle type dropper containing 20 drops/ml, or 50 microliters per drop.

For chickens which were administered Composition 1 as a feed additive, the feed additive was provided for a 4 day period. One group of 24 chickens were given a high dose of the feed additive. Another group of 24 chickens were given a medium dose, and another group of 24 chickens were given a low dose. Feed and water was provided ad libitum for 4 days, under routine conditions.

For chickens which were administered Composition 1 as a water additive, the water additive was provided for a 4 day period. One group of 24 chickens were given a high dose of the water additive. Another group of 24 chickens were given a medium dose, and another group of 24 chickens were given a low dose. Water was provided to the chickens ad libitum for 4 days, under routine conditions. Feed was be provided ad libitum. The control group in this experiment were housed and fed according to standard conditions for 4 days.

To determine the optimal effective dosage, each group of chickens were administered Composition 1 for a period of 1-4 days out of the week. The chickens were observed daily for any signs of general malaise or abnormal behavior, to include ruffled feathers, depressed posture, down birds, or any other indicators of stress or discomfort. Any lesions or abnormalities noted were recorded a daily basis. At the end of the study, the chickens were euthanized and individually necropsied to search for any lesions. Collected samples were placed in 10% formalin for histological assessment.

Feed and water were weighed and measured out per cage and per day. Amounts were be recorded on data collection forms. On Day 1 feed and water was measured and recorded for the first time. On Days 2-4, additional feed and water was measured and documented as added to the feed and water listing. On Day 5, the remaining feed and water were weighed, and the total feed and water consumed on a per cage basis for the entire study period was calculated.

Table 2 summarizes the change in body weight data. The differences among the treatment groups were not statistically significant for chickens which were given low feed, low water, low feed and water, medium feed, high feed, nasal and control.

TABLE 2
Body Weight (grams) Summary
Pen 18 Deleted

| | | Pre- | Post- | Change: |
|---|---|---|---|---|
| Treatment Group | Statistic | Treatment | Treatment | Post-Pre |
| Control | N BIRDS | 9 | 9 | 9 |
| | MEAN | 87.0 | 126.1 | 39.1 a |
| | MEDIAN | 88.7 | 126.0 | 38.2 |
| | STD | 5.2 | 6.4 | 2.6 |
| | MIN | 79.8 | 118.0 | 35.7 |
| | MAX | 94.6 | 135.0 | 43.6 |
| | P-VALUE | | | 0.0000 |
| High Feed | N BIRDS | 12 | 12 | 12 |
| | MEAN | 85.8 | 122.6 | 36.8 a |
| | MEDIAN | 85.0 | 119.5 | 36.1 |
| | STD | 8.8 | 11.3 | 4.9 |
| | MIN | 67.1 | 99.0 | 27.8 |
| | MAX | 97.5 | 139.0 | 43.7 |
| | P-VALUE | | | 0.0000 |
| High Feed and Water | N BIRDS | 12 | 12 | 12 |
| | MEAN | 90.5 | 88.3 | −2.1 c |
| | MEDIAN | 89.4 | 90.7 | −0.8 |
| | STD | 10.2 | 12.9 | 12.2 |
| | MIN | 78.4 | 66.0 | −17.7 |
| | MAX | 108.2 | 115.0 | 23.4 |
| | P-VALUE | | | 0.5536 |
| High Water | N BIRDS | 12 | 12 | 12 |
| | MEAN | 86.5 | 93.6 | 7.1 b |
| | MEDIAN | 87.4 | 91.3 | 10.0 |
| | STD | 7.6 | 25.1 | 22.2 |
| | MIN | 69.2 | 63.3 | −19.9 |
| | MAX | 97.6 | 141.0 | 49.0 |
| | P-VALUE | | | 0.2915 |
| Low Feed | N BIRDS | 12 | 12 | 12 |
| | MEAN | 92.7 | 136.7 | 44.0 a |
| | MEDIAN | 93.5 | 135.6 | 43.5 |
| | STD | 8.5 | 12.4 | 5.1 |
| | MIN | 75.9 | 111.3 | 35.4 |
| | MAX | 109.1 | 159.0 | 52.8 |
| | P-VALUE | | | 0.0000 |
| Low Feed and Water | N BIRDS | 12 | 12 | 12 |
| | MEAN | 84.4 | 121.3 | 36.9 a |
| | MEDIAN | 81.7 | 117.9 | 35.0 |
| | STD | 7.8 | 10.5 | 5.5 |
| | MIN | 74.1 | 108.2 | 30.8 |
| | MAX | 98.3 | 142.9 | 47.4 |
| | P-VALUE | | | 0.0000 |
| Low Water | N BIRDS | 12 | 12 | 12 |
| | MEAN | 90.3 | 131.7 | 41.4 a |
| | MEDIAN | 90.3 | 130.5 | 41.3 |
| | STD | 9.1 | 13.5 | 5.5 |
| | MIN | 74.5 | 112.7 | 32.4 |
| | MAX | 105.2 | 160.0 | 54.8 |
| | P-VALUE | | | 0.0000 |
| Medium Feed | N BIRDS | 12 | 12 | 12 |
| | MEAN | 86.8 | 127.5 | 40.7 a |
| | MEDIAN | 88.1 | 129.4 | 40.9 |
| | STD | 8.6 | 11.4 | 4.8 |
| | MIN | 71.2 | 108.5 | 31.4 |
| | MAX | 103.6 | 145.0 | 49.8 |
| | P-VALUE | | | 0.0000 |
| Medium Feed and Water | N BIRDS | 12 | 12 | 12 |
| | MEAN | 86.5 | 78.2 | −8.4 c |
| | MEDIAN | 86.0 | 70.4 | −14.8 |
| | STD | 8.8 | 21.4 | 13.9 |
| | MIN | 75.8 | 58.8 | −21.3 |
| | MAX | 101.5 | 120.4 | 18.9 |
| | P-VALUE | | | 0.0613 |
| Medium Water | N BIRDS | 12 | 12 | 12 |
| | MEAN | 84.6 | 74.1 | −10.5 c |
| | MEDIAN | 83.8 | 72.7 | −13.3 |
| | STD | 8.0 | 9.8 | 11.5 |
| | MIN | 74.2 | 63.0 | −22.6 |
| | MAX | 97.3 | 92.6 | 15.5 |
| | P-VALUE | | | 0.0089 |
| Nasal Drop | N BIRDS | 12 | 12 | 12 |
| | MEAN | 84.6 | 122.4 | 37.7 a |
| | MEDIAN | 87.7 | 123.6 | 37.5 |
| | STD | 11.5 | 16.2 | 6.1 |
| | MIN | 64.2 | 94.6 | 30.0 |
| | MAX | 97.7 | 146.8 | 49.1 |
| | P-VALUE | | | 0.0000 |

| | | |
|---|---|---|
| OVERALL P-VALUE FOR | 0.3145 | <0.0001 |
| TREATMENT GROUP | | |
| COMPARISON | | |

Table 3 summarizes the water and feed consumption data. In this table the data are summarized on a per pen basis. 4 cages that were not used were included in the analysis to show the naturally occurring feed and water loss. The differences among the treatment groups were not statistically significant for chickens which were given low feed, low water, low feed and water, medium feed, high feed, nasal and control groups.

TABLE 3
Feed and Water Consumption Summary
Pen 18 Deleted

| | | Feed | Water |
|---|---|---|---|
| Treatment | | Consumption | Consumption |
| Group | Statistic | (grams) | (mls) |
| CAGE NOT USED | N PENS | 4 | 4 |
| | MEAN | 1.8 d | 203.8 c |
| | MEDIAN | 1.5 | 189.5 |
| | STD | 1.4 | 30.9 |
| | MIN | 0.5 | 186.0 |
| | MAX | 3.7 | 250.0 |
| Control | N PENS | 3 | 3 |
| | MEAN | 194.3 a | 434.0 a |
| | MEDIAN | 192.1 | 427.0 |
| | STD | 4.3 | 26.2 |
| | MIN | 191.6 | 412.0 |
| | MAX | 199.3 | 463.0 |
| High F & W | N PENS | 4 | 4 |
| | MEAN | 78.4 bc | 296.0 b |
| | MEDIAN | 77.9 | 289.5 |
| | STD | 40.7 | 44.7 |
| | MIN | 38.9 | 253.0 |
| | MAX | 118.8 | 352.0 |
| High Feed | N PENS | 4 | 4 |
| | MEAN | 190.4 a | 417.8 a |
| | MEDIAN | 188.9 | 458.0 |
| | STD | 17.3 | 92.2 |
| | MIN | 172.1 | 280.0 |
| | MAX | 211.7 | 475.0 |
| High Water | N PENS | 4 | 4 |
| | MEAN | 97.9 b | 262.0 bc |
| | MEDIAN | 98.0 | 270.5 |
| | STD | 63.8 | 47.6 |
| | MIN | 27.7 | 202.0 |
| | MAX | 167.9 | 305.0 |
| Low F & W | N PENS | 4 | 4 |
| | MEAN | 183.0 a | 438.8 a |
| | MEDIAN | 178.9 | 446.5 |
| | STD | 9.1 | 40.0 |
| | MIN | 177.5 | 387.0 |
| | MAX | 196.5 | 475.0 |
| Low Feed | N PENS | 4 | 4 |
| | MEAN | 197.1 a | 437.8 a |
| | MEDIAN | 195.3 | 441.5 |
| | STD | 9.9 | 21.5 |
| | MIN | 187.0 | 410.0 |
| | MAX | 210.6 | 458.0 |
| Low Water | N PENS | 4 | 4 |
| | MEAN | 194.9 a | 418.3 a |
| | MEDIAN | 191.1 | 421.5 |
| | STD | 8.3 | 48.4 |
| | MIN | 190.0 | 365.0 |
| | MAX | 207.3 | 465.0 |
| Medium F & W | N PENS | 4 | 4 |
| | MEAN | 49.6 c | 277.0 b |
| | MEDIAN | 47.8 | 284.5 |
| | STD | 16.3 | 39.1 |
| | MIN | 33.7 | 228.0 |
| | MAX | 69.1 | 311.0 |
| Medium Feed | N PENS | 4 | 4 |
| | MEAN | 191.8 a | 447.3 a |
| | MEDIAN | 197.3 | 457.0 |
| | STD | 18.2 | 32.7 |
| | MIN | 165.8 | 400.0 |
| | MAX | 206.8 | 475.0 |
| Medium Water | N PENS | 4 | 4 |
| | MEAN | 51.9 c | 262.3 bc |
| | MEDIAN | 52.8 | 271.5 |
| | STD | 7.0 | 29.0 |
| | MIN | 42.9 | 220.0 |
| | MAX | 59.3 | 286.0 |
| Nasal Drops | N PENS | 4 | 4 |
| | MEAN | 181.6 a | 427.5 a |
| | MEDIAN | 186.5 | 435.5 |
| | STD | 12.1 | 36.1 |
| | MIN | 163.8 | 382.0 |
| | MAX | 189.4 | 457.0 |

| | | |
|---|---|---|
| OVERALL P-VALUE FOR | <0.0001 | <0.0001 |
| TREATMENT GROUP | | |
| COMPARISON | | |

Table 4 summarizes the ratio of weight gain to feed consumption data. In this table, the data are summarized on a per pen basis. The analysis revealed that the differences among the following treatment groups were not statistically significant: low feed, low water, low feed and water, medium feed, high feed, nasal and control.

TABLE 4
Body Weight (grams)/Feed Consumption Summary (grams)
Pen 18 Deleted

| | | Body |
|---|---|---|
| Treatment | | Weight/Feed |
| Group | Statistic | Consumption |
| Control | N PENS | 3 |
| | MEAN | 0.603 a |
| | MEDIAN | 0.600 |
| | STD | 0.016 |
| | MIN | 0.589 |
| | MAX | 0.620 |
| | P-VALUE | 0.0002 |
| High F & W | N PENS | 4 |
| | MEAN | −0.066 ab |
| | MEDIAN | −0.159 |
| | STD | 0.568 |
| | MIN | −0.638 |
| | MAX | 0.690 |
| | P-VALUE | 0.8303 |
| High Feed | N PENS | 4 |
| | MEAN | 0.582 a |
| | MEDIAN | 0.582 |
| | STD | 0.056 |
| | MIN | 0.523 |
| | MAX | 0.640 |
| | P-VALUE | 0.0002 |
| High Water | N PENS | 4 |
| | MEAN | −0.254 b |
| | MEDIAN | 0.199 |
| | STD | 1.181 |
| | MIN | −2.004 |
| | MAX | 0.590 |
| | P-VALUE | 0.6963 |
| Low F & W | N PENS | 4 |
| | MEAN | 0.604 a |
| | MEDIAN | 0.622 |
| | STD | 0.044 |
| | MIN | 0.539 |
| | MAX | 0.633 |
| | P-VALUE | 0.0001 |
| Low Feed | N PENS | 4 |
| | MEAN | 0.669 a |
| | MEDIAN | 0.670 |
| | STD | 0.025 |
| | MIN | 0.645 |
| | MAX | 0.691 |
| | P-VALUE | 0.0000 |
| Low Water | N PENS | 4 |
| | MEAN | 0.638 a |
| | MEDIAN | 0.638 |
| | STD | 0.008 |
| | MIN | 0.627 |
| | MAX | 0.646 |
| | P-VALUE | 0.0000 |
| Medium F & W | N PENS | 4 |
| | MEAN | −0.708 b |
| | MEDIAN | −0.778 |
| | STD | 0.843 |
| | MIN | −1.474 |
| | MAX | 0.197 |
| | P-VALUE | 0.1916 |
| Medium Feed | N PENS | 4 |
| | MEAN | 0.636 a |
| | MEDIAN | 0.630 |
| | STD | 0.027 |
| | MIN | 0.610 |
| | MAX | 0.674 |
| | P-VALUE | 0.0000 |
| Medium Water | N PENS | 4 |
| | MEAN | −0.659 b |
| | MEDIAN | −0.509 |
| | STD | 0.556 |
| | MIN | −1.415 |
| | MAX | −0.203 |
| | P-VALUE | 0.0985 |
| Nasal Drops | N PENS | 4 |
| | MEAN | 0.623 a |
| | MEDIAN | 0.629 |
| | STD | 0.015 |
| | MIN | 0.600 |
| | MAX | 0.634 |
| | P-VALUE | 0.0000 |

These results provide suitable dosing for Composition 1 for poultry. The data demonstrates that Composition 1 can be provided to growing poultry in a medicated feed at various (low, medium, and high) concentrations, without safety issues or tolerability problems.

### Example 3

The undiluted antiviral composition of Example 1, Composition 1 and 10-fold serial dilutions were prepared and tested for antiviral activity against the IBV in VERO E6 cells and in 10-day old embryonating chicken eggs. In addition, a placebo was similarly diluted and tested.

The continuous cell line VERO E6 (CRL-1586) was obtained from the American Type Culture Collection (Rockdale, Md.) and propagated in Minimum Essential Medium (Eagle) with 2 mM L-glutamine, 1.5 g/L sodium bicarbonate, 0.1 mM non-essential amino acids, 1.0 mM sodium pyruvate, and 10% fetal bovine serum (Invitrogen Corp [Gibco], Carlsbad, Calif.) at 37 C and 5% CO₂. The cells were grown in a T75 flask (BD Biosciences, Franklin Lakes, N.J.) and transferred to 96 well plates and grown to 90% confluence.

A 1×10³ concentration of Beaudette strain of IBV, a tissue culture infectious dose₅₀ (TCID₅₀), was mixed with seven 10-fold serial dilutions (beginning with a dilution of 1×10⁻³, which is nontoxic for the cells) of the antiviral compound or the placebo prepared in cell culture maintenance media (containing 1% fetal bovine serum). The mixtures were incubated at room temperature for 30 minutes; then nine 10-fold serial dilutions of each mixture were prepared for inoculation onto cells. The cell culture media was removed from the cells and the mixtures were inoculated onto the monolayers. Negative control wells receiving cell culture maintenance media only were also included in the experiment. The cells were incubated for 7 days at 37 C and 5% CO₂ and examined twice daily for cytopathic effects (CPE).

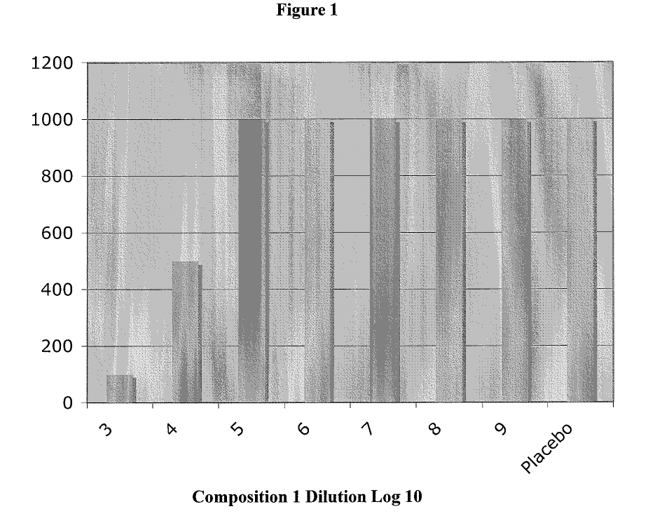

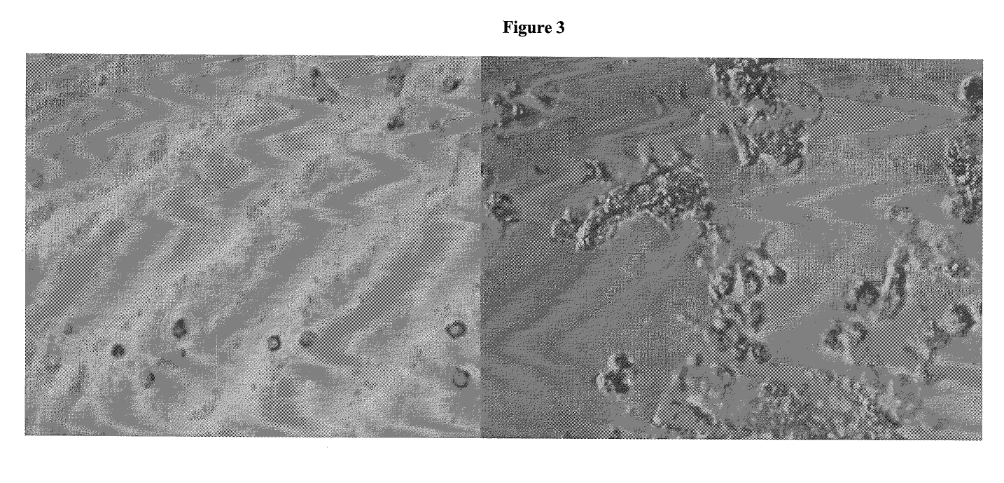

FIG. 1 shows the results of the antiviral affect on IBV. FIG. 3 shows VERO E6 cells protected from viral infection by the composition of the present invention and cells, to which the composition had not been applied, infected with IBV. The antiviral compound of Composition 1 at a 1×10⁻³ dilution reduced the titer of IBV over 100-fold, from an average of 1×10³⁰ to 1×10¹⁸ TCID₅₀. Composition 1 at a dilution of 1×10⁻⁴ reduced the IBV titer 2-fold. None of the other higher dilutions of Composition 1 reduced the titer of IBV. By comparison, no reduction in virus titer for IBV was observed for the placebo at any dilution, indicating that the active ingredient in Composition 1 was responsible for the antiviral effect. Additionally, none of the negative control wells had CPE.

Embryonic chicken eggs, infected with IBV, were also tested to determine the efficacy of the composition. The embryonic chicken eggs were prepared in the same fashion as that of the VERO E6 cells. The experimental design was the same, except 10-day old embryonating chicken eggs were inoculated instead of tissue culture cells and the beginning concentration of Composition 1 and the placebo was undiluted since the compounds are not toxic for the embryos. Specific pathogen free (SPF) fertile chicken eggs were obtained from Sunrise farms (Catskill, N.Y.) and incubated at 37 C for 10 days. The embryonated eggs were inoculated into the chorioallantoic sac (CAS) with 200 ul of undiluted and each of the 10-fold dilutions of the composition or the placebo prepared in PBS (pH 7.4) and mixed with either 1×10⁴ embryo infectious dose₅₀ (EID₅₀) of 1×10⁷ EID₅₀ of IBV. Negative control eggs that received PBS only were also included. The eggs were incubated at 37 C and candled daily for 7 days to record mortality. Any mortality occurring within the first 24 hours was considered to be due to trauma associated with inoculation and disregarded. On the 7^{th} day, all the remaining eggs were chilled to 4 C and opened to examine the embryos for clinical signs.

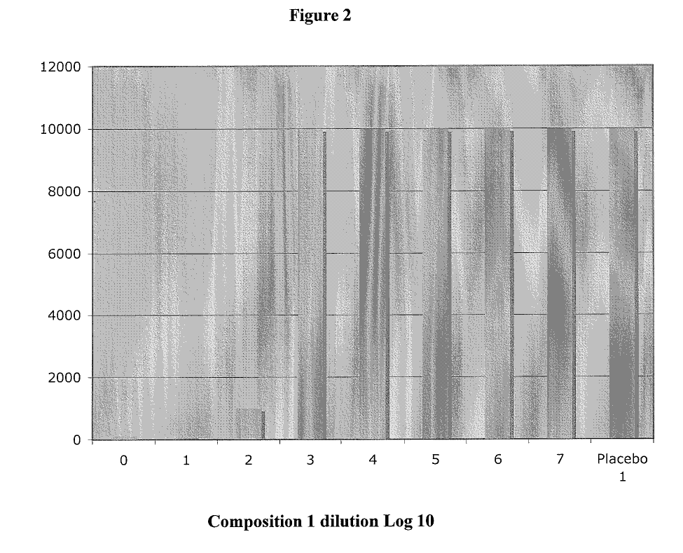

FIG. 2 shows the affect of Composition 1 on IBV in embryonating eggs. Dilutions of Composition 1 out to 1×10⁻² had a measurable affect on the titer of IBV, reducing it over 100-fold. By comparison, no affect was observed for dilutions of 1×10⁻³ to 1×10⁻⁷ or for the placebo at any dilution, indicating that the active ingredient in Composition 1 was responsible for the antiviral effect.

### Example 4

### Testing Against Infectious Bronchitis Virus in Chickens

The timing of treatment was 6 hours before challenge, 2 hours before challenge and 2 hours after challenge. Antiviral activity was assessed by examining the birds for infectious bronchitis virus (IBV) using real-time RT-PCR at 5 days post-challenge and by recording clinical signs and lesions characteristic of the disease.

2-week old specific pathogen free leghorn chickens were used for this study. The birds were maintained in HEPA filtered positive pressure horsefal isolation units and given feed and water ad libitum.

3 different routes of administering the composition were tested. For the intranasal route 200 ul of the composition of Example 1 was applied directly to the nares of each bird.

A nebulizer was used to spray approximately 1 ml of the composition of Example 1 per bird. The fresh air delivery to the isolator was temporarily blocked, the birds were then sprayed, and approximately 10 minutes later, fresh air delivery was resumed.

Because birds have a cleft pallet, delivering the composition in the drinking water ought to expose the drug to the oral cavity and sinuses. The birds were water starved for 1 hour prior to delivery of the composition of Example 1 in the drinking water. Approximately 1 ml of the composition of Example 1 per bird was mixed with and equal volume of reconstituted non-fat dry milk. If the birds did not drink all of the liquid within 30 minutes, the birds were orally dosed with equal volumes of the remaining material. After the antiviral compound was consumed, fresh water was provided.

TABLE 5
Number of Birds in Each Group.

| Groups: | 6 hrs | 2 hrs | 2 hrs | No |
|---|---|---|---|---|
| (treatment/challenged) | BC | BC | PC | treatment |
| Intranasal/challenged | 10 | 10 | 10 | |
| Spray/challenged | 10 | 10 | 10 | |
| Water/challenged | 10 | 10 | 10 | |
| None/Challenged | | | | 10 |
| None/not challenged | | | | 10 |

BC = before challenge
PC = post = challenge

All of the treated birds and one of the untreated groups were challenged with 1×10⁴ embryo infectious dose₅₀ of the Mass 41 pathogenic strain of IBV per bird. The challenge virus was administered intranasally and the birds were examined twice daily for clinical signs of the disease. This level of challenge virus was sufficient to infect 90% of the birds as determined by virus detection in the trachea using real-time RT-PCR.

Clinical signs were recorded daily and any moribund birds were removed, killed and necropsied. At 5 days post-challenge (this is convention for IBV vaccine efficacy testing) the birds were killed and necropsied. At necropsy, sinus and tracheal swabs were collected and placed in ice cold PBS (pH 7.4). The presence of virus was determined by quantitative real-time RT-PCR directly from sinus and tracheal swabs. Efficacy was based on not less than 90% of the non-treated controls positive for challenge virus and not less than 90% of the treated birds negative for virus. Efficacy was also based on clinical signs and lesions. Lesions were recorded and tracheas were harvested for histopathology. Tracheas were collected in 10% neutral buffered formalin and submitted for histopathology. The tissues were routinely processed into paraffin, and 5-μm sections were cut for hematoxylin and eosin staining. Epithelial hyperplasia, lymphocyte infiltration, and the severity of epithelial deciliation were scored for each trachea.

All of the treated/challenged groups were compared to the control groups (both challenged and non-challenged).

Intranasal inoculation either 2 hours before or 2 hours after challenge and spray at 6 hours before, 2 hours before and 2 hours after challenge gave the best results. A summary of the data is presented below in Table 6.

TABLE 6
Clinical signs and virus detection in 2 week old chickens following
treatment with the composition of Example 1 before and after
infectious bronchitis virus (IBV) challenge^{a}

| | | Virus | Average Ct |
|---|---|---|---|
| Group | Clinical Signs^{b} | Detection^{c} | Value^{d} |
| Intranasal 6 hours BC | 9/10 | 10/10 | 23.48 |
| Intranasal 2 hours BC | 6/10 | 0/10 | Neg |
| Intranasal 2 hours PC | 7/10 | 0/10 | Neg |
| Spray 6 hours BC | 7/10 | 0/10 | Neg |
| Spray 2 hours BC | 7/10 | 0/10 | Neg |
| Spray 2 hours PC | 7/10 | 0/10 | Neg |
| Water 6 hours BC | 10/10 | 9/10 | 23.13 |
| | | | (avg of 9 pos) |
| Water 2 hours BC | 10/10 | 10/10 | 23.31 |
| Water 2 hours PC | 10/10 | 10/10 | 24.72 |
| Challenge control | 10/10 | 10/10 | 26.01 |
| Negative control | 0/10 | 0/10 | Neg |

^{a}The birds were intranasally challenged with 3.1 × 10⁴ embryo infectious dose₅₀/bird of pathogenic IBV strain Mass41.
^{b}Clinical signs were recorded 5 days following challenge and consisted of tracheal rales, watery eyes and in some cases (challenge control only) mucus in the naris.
^{c}Virus was detected directly from tracheal swabs collected 5 days following challenge by real time RT-PCR.
^{d}The average cycle threshold (Ct) value for the real time RT-PCR test indicates the relative amount of virus detected in the trachea.

### Example 5

### Dose Response Experiment

Spray inoculation was used to identify the least amount of active composition capable of eliciting an antiviral affect against IBV in chickens.

The same type and age of birds, and housing used in Example 4 were also used in this example.

The following concentrations of the formulation of Example 1 were used: undiluted, and diluted with water at the ratios of formulation:water of: 1:5, 1:10, 1:20, 1:40, 1:80, 1:160, 1:320, 1:640, and 1:1280. The treated birds and one of the untreated groups of birds were challenged as described in Example 4. The other untreated group served as the negative control group.

TABLE 7
Experimental protocol showing dilutions, treatments and number of
birds for each group^{a}.

| Treatment | Undiluted | 1:5 | 1:10 | 1:20 | 1:40 | 1:80 | 1:160 | 1:320 | 1:640 | 1:1280 |
|---|---|---|---|---|---|---|---|---|---|---|
| Spray 2 | 10 | 10 | 10 | 10 | 10 | 10 | 10 | 10 | 10 | 10 |
| hours | | | | | | | | | | |
| before | | | | | | | | | | |
| Spray 2 | 10 | 10 | 10 | 10 | 10 | 10 | 10 | 10 | 10 | 10 |
| hours | | | | | | | | | | |
| after | | | | | | | | | | |

^{a}Additional control groups not included in the table are;
1. 10 birds treated by the intranasal route with undiluted formulation of Example 1 and not challenged,
2. 10 birds that were treated by spray inoculation with undiluted formulation of Example 1 and not challenge.
3. 10 birds that were not treated and were challenged,
4. 10 birds that served as an untreated, unchallenged negative control.

The procedures of Example 4 were followed for data collection and analysis except that the histopathological analysis was not conducted. The results are given in Table 8.

Treatment 2 hours before challenge lowered the detectable virus by 3 on a logarithmic scale, whereas; treatment 2 hours after challenge lowered the detectable virus by 2.5 on a logarithmic scale. This is extremely significant since it is known that challenge with less than 1×10³ EID₅₀ of pathogenic IBV in 2-week old susceptible specific pathogen free chickens does not result in disease in most of the birds. This is consistent with the data below where fewer birds showed clinical signs in the treated groups at the lower dilutions. Thus, treatment 2 hours before challenge with the compositions of the invention at a 1:40 dilution or less would likely lessen or prevent disease in the field. Treatment 2 hours after infection showed a consistent dose response and likely results in amelioration of disease when given at a 1:40 dilution or less.

TABLE 8
Dose titration of spray treatment in 2 week old specific pathogen free
chickens 2 hours before or 2 hours after challenge with pathogenic IBV.

| | Average Ct value ± | | Average Ct value ± | |
|---|---|---|---|---|
| | SD 2 hours | | SD 2 hours after | |
| | before challenge | # of Birds | challenge | # of Birds |
| | (approximate | with Clinical | (approximate | with Clinical |
| Treatment | genome copy #) | signs/total | genome copy #) | signs/total |
| Undiluted + | 25.8 ± 1.1^{A} (10) | 6/10 | 21.3 ± 2.3^{A} (50) | 3/10 |
| Challenge | | | | |
| 1:5 + Challenge | 26.1 ± 1.3^{A} (10) | 6/10 | 20.1 ± 2.8^{AB} (500) | 6/10 |
| 1:10 + Challenge | 25.5 ± 1.5^{A} (10) | 3/10 | 21.4 ± 2.0^{A} (50) | 7/10 |
| 1:20 + Challenge | 23.5 ± 1.4^{A} (50) | 4/10 | 20.8 ± 1.6^{AB} (500) | 6/10 |
| 1:40 + Challenge | 19.7 ± 1.3^{AB} (500) | 8/10 | 21.1 ± 1.4^{A} (50) | 5/10 |
| 1:80 + Challenge | 20.3 ± 1.9^{AB} (500) | 6/10 | 19.7 ± 1.3^{AB} (1,000) | 6/10 |
| 1:160 + Challenge | 19.7 ± 1.6^{AB} (500) | 4/10 | 19.1 ± 1.7^{AB} (1,000) | 9/10 |
| 1:320 + Challenge | 20.7 ± 2.9^{AB} (500) | 5/10 | 19.7 ± 2.3^{AB} (1,000) | 6/10 |
| 1:640 + Challenge | 18.6 ± 1.2^{C} (10,000) | 7/10 | 18.6 ± 0.9^{B} (10,000) | 9/10 |
| 1:1280 + | 20.2 ± 1.6^{AB} (500) | 8/10 | 18.5 ± 0.3^{B} (10,000) | 8/10 |
| Challenge | | | | |
| No treatment + | 18.9 ± 1.1^{C} (10,000) | 9/10 | 20.8 ± 1.3^{AB}/7 birds | 7/7 |
| Challenge | | | (500)* | |
| Undiluted/No | 40 ± 0.0^{C} (0) | 0/10 | ND | ND |
| challenge | | | | |
| No treatment/No | 39.6 ± 0.8^{C} (0) | 0/10 | ND | ND |
| challenge | | | | |

Treatment = Formulation of Example 1 sprayed undiluted or diluted at a dose of 1 ml/bird followed by challenge with 1 × 10^{4.5} EID₅₀ of pathogenic IBV, Mass41 strain.
Numbers within a column with different lettered superscripts are statistically different at p < 0.1 (Tukey-Kramer HSD)
Avg Ct represents tracheal swabs from 10 birds per group, unless indicated otherwise, with swabs taken 5 days after challenge.
Approximate genome copy number = number of viral genomes detected in the tracheal swab based on a standard curve of known viral genome copy numbers.
*Likely this value is not accurate since 3 birds died in this group. It could not be determined if the mortality was due to challenge.
ND = Not done.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

1. A method for treatment of a bird having an infectious bronchitis viral strain, comprising the step of administering to a bird that has the infectious bronchitis viral strain, a safe and effective amount of composition comprising:
a first ingredient selected from the group consisting of turmeric powder extract, turmeric fluid extract, turmeric extract, turmeric powder, at least a part of a whole plant of turmeric, a turmeric tincture, and mixtures thereof;
a second ingredient selected from the group consisting of green tea powder, green tea powder extract, green tea fluid extract, at least a part of a whole plant of green tea, tinctures of green tea and mixtures thereof;
a third ingredient selected from the group consisting of ginger powder extract, ginger fluid extract, ginger powder, at least a part of a whole plant of ginger, a ginger tincture and mixtures thereof, and
an acceptable carrier;
said amount being effective, when administered, to reduce an incidence of other birds contracting infectious bronchitis.

2. A method for the prophylactic use of a composition to reduce an incidence or transmissivity of infectious bronchitis, comprising the step of administering to a bird that has been, or will be, exposed to infectious bronchitis, a safe and effective amount of a composition comprising:
a first ingredient selected from a group consisting of turmeric powder extract, turmeric fluid extract, turmeric extract, turmeric powder, at least a part of a whole plant of turmeric, a turmeric tincture, and mixtures thereof;
a second ingredient selected from the group consisting of green tea powder, green tea powder extract, green tea fluid extract, at least a part of a whole plant of green tea, tinctures of green tea and mixtures thereof;
a third ingredient selected from the group consisting of ginger powder extract, ginger fluid extract, ginger powder, at least a part of a whole plant of ginger, a ginger tincture and mixtures thereof; and
an acceptable carrier;
said amount being effective, when administered, to reduce the incidence of infectious bronchitis in said bird or to reduce an incidence of infectious bronchitis in other birds exposed to said treated bird.

3. The method of claim 1, wherein the composition is administered in a form selected from a group consisting of a dry formulation, a liquid formulation, a tablet, a capsule, feed additive and water additive.

4. The method of claim 1, wherein the composition is administered as an aerosol, a spray or a mist.

5. The method of claim 1, wherein the composition is administered in ovo.

6. The method of claim 1, wherein administering the composition further comprises the step of using an effective amount of the composition to disinfect an item of equipment so as to render inactive at least some infectious bronchitis virus located on the equipment.

7. The method of claim 1, wherein the first ingredient comprises turmeric extract and the second ingredient comprises green tea extract.

8. The method of claim 1, wherein each gram of the composition contains about 0.001 mg to about 20 mg of green tea extract, and about 0.0011 mg to about 30 mg of turmeric powder extract.

9. The method of claim 1, wherein the third ingredient comprises ginger extract.

10. The method of claim 9, wherein the composition contains about 0.001 mg to about 30 mg of ginger extract.

11. The method of claim 1, wherein the composition further, comprises a fourth ingredient selected from the group consisting of horseradish root, horseradish oil, horseradish powder extracts, horseradish fluid extracts and horseradish root extracts.

12. The method of claim 1, wherein the composition further comprises one or more ingredients selected from the group consisting of ethanol, propylene glycol, glycerine, phospholipids, medium chain triglyceride oil and water.

13. The method of claim 11, wherein the fourth ingredient comprises a horseradish root extract.

14. A bird feed which comprises:
a bird food component, and
a safe and effective amount of a composition comprising:
a first ingredient selected from a group consisting of turmeric powder extract, turmeric fluid extract, turmeric extract, turmeric powder, at least a part of a whole plant of turmeric, a turmeric tincture, and mixtures thereof;
a second ingredient selected from the group consisting of green tea powder, green tea powder extract, green tea fluid extract, at least a part of a whole plant of green tea, tinctures of green tea and mixtures thereof; and
a third ingredient selected from the group consisting of ginger powder extract, ginger fluid extract, ginger powder, at least a part of a whole plant of ginger, a ginger tincture and mixtures thereof; and
said amount being effective, when administered as a feed to a bird, to treat infectious bronchitis, reduce the incidence of infectious bronchitis in said bird, or to reduce an incidence of infectious bronchitis in other birds exposed to said treated bird.

15. The bird feed of claim 14, wherein the first ingredient comprises turmeric extract and the second ingredient comprises green tea extract.

16. The bird feed of claim 14, wherein each gram of the composition contains about 0.001 mg to about 20 mg of green tea extract, and about 0.001 mg to about 30 mg of turmeric powder extract.

17. The bird feed of claim 15, wherein the third ingredient comprises ginger extract.

18. The bird feed of claim 16, wherein the composition contains about 0.001 mg to about 30 mg of ginger extract.

19. The bird feed of claim 14, wherein the composition further comprises a fourth ingredient selected from the group consisting of horseradish oil, horseradish root, horseradish powder extracts, horseradish fluid extracts and horseradish root extracts.

20. The bird feed of claim 14, wherein the composition further comprises one or more ingredients selected from the group consisting of ethanol, propylene glycol, glycerine, phospholipids, medium chain triglyceride oil and water.

21. The bird feed of claim 14, wherein the bird feed contains about 0.001 to about 50 weight percent of the composition, based on the total weight of the bird feed.

22. The bird feed of a claim 14, wherein the bird feed comprises about 0.01 to about 30 weight percent of the composition, based on the total weight of the bird feed.

23. The bird feed of claim 14, wherein the bird feed comprises about 0.1 to about 20 weight percent of the composition, based on the total weight of the bird feed.

24. The bird feed of claim 19, wherein the fourth ingredient comprises horseradish oil.

25. The method of claim 2, wherein the composition is administered in a form selected from a group consisting of a dry formulation, a liquid formulation, a tablet, a capsule, feed additive and water additive.

26. The method of claim 2, wherein the composition is administered as an aerosol, a spray or a mist.

27. The method of claim 2, wherein the composition is administered in ovo.

28. The method of claim 2, wherein administering the composition further comprises the step of using an effective amount of the composition to disinfect an item of equipment so as to render inactive at least some Newcastle disease virus located on the equipment.

29. The method of claim 2, wherein the first ingredient comprises turmeric extract and the second ingredient comprises green tea extract.

30. The method of claim 2, wherein each gram of the composition contains about 0.001 mg to about 20 mg of green tea extract, and about 0.001 mg to about 30 mg of turmeric powder extract.

31. The method of claim 29, wherein the third ingredient comprises ginger extract.

32. The method of claim 31, wherein the composition contains about 0.001 mg to about 30 mg of ginger extract.

33. The method of claim 2, wherein the composition further comprises a fourth ingredient selected from the group consisting of horseradish oil, horseradish powder extracts, horseradish fluid extracts and horseradish root extracts.

34. The method of claim 2, wherein the composition further comprises one or more ingredients selected from the group consisting of ethanol, propylene glycol, glycerine, phospholipids, medium chain triglyceride oil and water.

35. The method of claim 33, wherein the composition comprises horseradish oil.
